(19) United States
(12) Patent Application Publication
(10) Pub. No.: US 2016/0077169 A1
(43) Pub. Date: Mar. 17, 2016

(54) System and Method for Coordinating Use of An Interventional or Implantable Device With External Magnetic Fields

(71) Applicant: The General Hospital Corporation, Boston, MA (US)

(72) Inventors: Ouri Cohen, Brookline, MA (US); Jerome L. Ackerman, Newton, MA (US)

(21) Appl. No.: 14/853,662

(22) Filed: Sep. 14, 2015

Related U.S. Application Data
(60) Provisional application No. 62/050,404, filed on Sep. 15, 2014.

(51) Int. Cl. G01R 33/28; A61B 5/055

(52) U.S. Cl. CPC G01R 33/288; G01R 33/285; A61B 5/055

(57) ABSTRACT

A system and method is provided for use with a magnetic resonance imaging (MRI) system to facilitate use of a medical device during operation of the MRI system. The system includes a probe configured to be electrically coupled to a medical device configured to be inserted or implanted into a subject and an analyzer system configured to receive data from the probe and determine resonant frequencies of the medical device using the data. The system also includes a display configured to generate a report of the resonant frequencies of the medical device at least over predicted operating frequencies of the MRI system during an imaging procedure using the MRI system and in the presence of the medical device and indicating any resonant frequencies of the medical device coincident with the predicted operating frequencies of the MRI system during the medical procedure.

### CROSS-REFERENCE TO RELATED APPLICATIONS

The present application is based on, claims priority to, and incorporates herein by reference in its entirety, U.S. Provisional Application Ser. No. 62/050,404, filed Sep. 15, 2014, and entitled “Magnetic Resonance Safety Device and Methods of Using the Same.”

### BACKGROUND

The present disclosure relates to systems and methods for medical imaging and, more particularly, to system sand methods for determining and/or controlling the resonant frequencies of an interventional or implanted device that is designed to be placed within the human body, despite influences of configuration.

When a substance such as human tissue is subjected to a uniform magnetic field (polarizing field B₀), the individual magnetic moments of the nuclear spins in the tissue tend to align with this polarizing field. If they are not initially aligned precisely with the polarizing field, they will precess about the field at their characteristic Larmor frequency as a top precesses about the Earth's gravitational field if the top's spin axis is not initially aligned with the field. Usually the nuclear spins are the nuclei of hydrogen atoms, but NMR active nuclei of other elements are occasionally used. At equilibrium, the individual magnetic moments of all the nuclei combine to produce a net magnetic moment M_{z} in the direction of the polarizing field, but the randomly oriented magnetic components in the perpendicular, or transverse, plane (x-y plane) cancel one another. If, however, the substance, or tissue, is subjected to a magnetic field (excitation field B₁; also referred to as the radiofrequency (RF) field) which is in the x-y plane and which oscillates near the Larmor frequency, the net aligned moment, M_{z}, may be rotated, or “tipped”, into the x-y plane to produce a net transverse magnetic moment Mₜ, which precesses (rotates about the B₀ field direction) in the x-y plane at the Larmor frequency. The typically brief application of the B₁ field that accomplishes the tipping of the nuclear spins is generally known as an RF pulse. The practical value of this phenomenon resides in the signal which is emitted by the excited spins after the excitation field B₁ is terminated. There is a wide variety of measurement pulse sequences (“sequences”) in which this nuclear magnetic resonance (“NMR”) phenomenon is exploited.

When utilizing these signals to produce images, the phenomenon is generally known as magnetic resonance imaging (“MRI”), and magnetic field gradients (Gₓ, G_{y}, and G_{z}) of the polarizing field B₀ are employed. Typically, the region to be imaged experiences a sequence of measurement cycles in which these gradients vary according to the particular localization method being used. The emitted MRI signals are detected using a receiver coil. The MRI signals are then digitized and processed to reconstruct the image using one of many well-known reconstruction techniques.

The lack of ionizing radiation and the ability to provide anatomical images of soft tissue with sufficient resolution makes MRI an appealing modality to couple with interventional medical procedures that can be performed less invasively or more efficiently or safely when guided by MR images. For example, the guidance of therapeutic devices, such as catheters, and/or the placement of interventional devices, such as guidewires and stents, using MRI guidance is a promising and evolving field with great clinical potential.

One particular challenge of this field, however, has been how to develop safe and reliable methods for tracking such devices as they are moved and manipulated within vessels or organs. The tips of guidewires can be easily visualized using conventional x-ray fluoroscopy by applying small, radio-opaque markers to the tips. Of course, x-ray fluoroscopy has the noted drawback of subjecting the patient and clinician to ionizing radiation. In MRI, the analog to the passive radio-opaque marker is a passive marker made of a material with a sufficiently large magnetic susceptibility, relative to the surrounding tissues, such as a stainless steel tip on a nitinol wire. In MR images depicting a guidewire containing such passive markers, a local hypointense region is present in the tissues adjacent to the markers, thereby resulting in a loss of clinically relevant information. Unfortunately, the high magnetic susceptibility of the material may induce artifacts in the MR images, among other drawbacks.

Accordingly, some other passive systems have been developed that have dedicated “indicator elements,” for example, including a paramagnetic material. In such devices a paramagnetic material may be integrated with a catheter or other interventional medical device to influence the magnetic resonance image of a patient to be examined by means of an MRI system. The influence the device has on MR images makes it possible to determine the position of the interventional instrument within the body of the patient without the instrument being directly visible. However, the influence of the paramagnetic component in the device on the MR image adversely affects the diagnostic quality of the magnetic resonance image. As a result of the influence of the indicator element, MR images will exhibit degraded or lost anatomical details in the regions adjacent to the indicator element.

Other methods to visualize interventional devices in patients have been developed, including affixing to the devices MRI-visible markers containing substances that appear in the MR images, and using the devices as if they are MRI receiver coils that detect MRI signals from the surrounding tissues along their length. These methods can similarly introduce artifacts into the MR images.

To further complicate matters, whenever a medical device, instrument, or implant containing electrically conductive components, especially long wires, is present during MRI scanning, there is a risk that the device could be heated when subjected to RF energy emitted by the MRI system. Whether the device is part of an interventional surgical procedure or a permanent implant, an whether the conductive component is ferromagnetic or nonferromagnetic this potential for significant heating exists, and can lead to device damage or destruction, and most seriously, to tissue damage and harm to the subject. The greatest risk generally occurs for long metallic wires, such as interventional guidewires, and electrical leads or electrodes of implanted medical devices such as cardiac pacemakers.

Previous works have shown that, under certain circumstances, an interventional medical device can inadvertently form a tuned electrical circuit that resonates with the RF field of an MRI scanner, which can cause the temperature proximate the device to rise. Some studies have show that the temperature rise can be upwards of 60° C., which can cause substantial tissue damage. Unfortunately, the resonant frequency of such an interventional device is dependent on a large number of variables that are difficult to both predict and control in-vivo. As such, some research groups have proposed replacing simple wires with complex, transmission lines that are designed to suppress the resonance effect. However, these transmission lines are bulky and complex compared to simple wires. Aside from their incompatibility with current interventional medical devices, it is not clear that these transmission lines can be made to have the required mechanical properties to serve as, for example, guidewires.

It would therefore be desirable to have a system and method for tracking interventional devices using MRI while controlling the response of such devices to RF energy from the MRI system.

### SUMMARY

The present disclosure overcomes the aforementioned drawbacks by providing systems and methods to measure the resonant frequencies of an interventional or implanted device that is designed to be placed within the human body, despite influences of configuration. The disclosure further provides systems and methods to alter the resonant frequencies of the device to control resonance with the RF field of an MRI scanner, thereby controlling any potential desired or undesired heating associated with the device.

In accordance with one aspect of the disclosure, a system is disclosed that is configured to be utilized with a magnetic resonance imaging (MRI) system to facilitate use of a medical device during operation of the MRI system. The system includes a probe configured to be electrically coupled to a medical device configured to be inserted or implanted into a subject and an analyzer system configured to receive data from the probe and determine resonant frequencies of the medical device using the data. The system also includes a display configured to generate a report of the resonant frequencies of the medical device at least over predicted operating frequencies of the MRI system during an imaging procedure using the MRI system and in the presence of the medical device and indicating any resonant frequencies of the medical device coincident with the predicted operating frequencies of the MRI system during the medical procedure.

In accordance with another aspect of the disclosure, a system is provided that is configured to be utilized with a magnetic resonance imaging (MRI) system to facilitate use of a medical device during operation of the MRI system. The system includes a probe configured to be electrically coupled to a medical device configured to be inserted or implanted into a subject to perform a medical procedure and an analyzer system configured to receive data from the probe and determine resonant frequencies of the medical device using the data. The system also includes a processor configured to determine the resonant frequencies of the medical device and compare the resonant frequencies of the medical device to predicted operating frequencies of the MRI system during the medical procedure. Upon determining a commonality of the resonant frequencies of the medical device during the medical procedure and the predicted operating frequencies of the MRI system, the processor is configured to adjust the resonant frequencies of the medical device to reduce the commonality of the resonant frequencies of the medical device during the medical procedure and the predicted operating frequencies of the MRI system.

In accordance with yet another aspect of the disclosure, a method is provided for performing an image guided interventional medical procedure. The method includes electrically analyzing a medical device to determine resonant frequencies of the medical device and comparing the resonant frequencies of the medical device to predicted operating frequencies of a magnetic resonance imaging (MRI) system during a planned medical procedure performed using the medical device and guided by images acquired by the MRI system. The method also includes determining a commonality of the resonant frequencies of the medical device during the medical procedure and the predicted operating frequencies of the MRI system and adjusting the resonant frequencies of the medical device to reduce the commonality of the resonant frequencies of the medical device during the medical procedure and the predicted operating frequencies of the MRI system. The method further includes arranging a subject within the MRI system to acquire images of the subject during the medical procedure performed using the medical device.

In accordance with still another aspect of the disclosure, a magnetic resonance imaging (MRI) system is provided. The MRI system includes a magnet system configured to generate a polarizing magnetic field about at least a portion of a subject arranged in the MRI system, a plurality of gradient coils configured to establish at least one magnetic gradient field to the polarizing magnetic field, and a radio frequency (RF) system configured to apply an RF field to the subject during a transmit phase and receive imaging signals from the subject during a receive phase. The MRI system also includes a processor configured to i) compare measured resonant frequencies of a medical device to predicted operating frequencies of the MRI system during a planned medical procedure performed using the medical device and guided by images acquired by the MRI system, ii) determine a commonality of the resonant frequencies of the medical device during the medical procedure and the predicted operating frequencies of the MRI system, and iii) adjust the resonant frequencies of the medical device to reduce the commonality of the resonant frequencies of the medical device during the medical procedure and the predicted operating frequencies of the MRI system. The processor is further configured to iv) repeat i) through iii) during the medical procedure.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a block diagram of an example of a magnetic resonance imaging (“MRI”) system.

FIG. 2 is a block diagram of an example of a radio frequency (“RF”) system that may form a part of the MRI system of FIG. 1.

FIG. 3 is a schematic diagram of a system for analyzing and/or adjusting resonant frequencies of a device in accordance with the present disclosure.

FIG. 4 is a schematic diagram of another system for analyzing and/or adjusting resonant frequencies of a device in accordance with the present disclosure.

FIG. 5 is an example of an analysis display or report that may be generated by an analyzing system of FIG. 3 or 4 in accordance with the present disclosure.

FIG. 6 is another example of a analysis display or report that may be generated by an analyzing system of FIG. 3 or 4 in accordance with the present disclosure.

FIG. 7 is still another example of an analysis display or report that may be generated by an analyzing system of FIG. 3 or 4 in accordance with the present disclosure.

FIG. 8 is a flow chart setting forth examples of steps for a process in accordance with the present disclosure.

### DETAILED DESCRIPTION

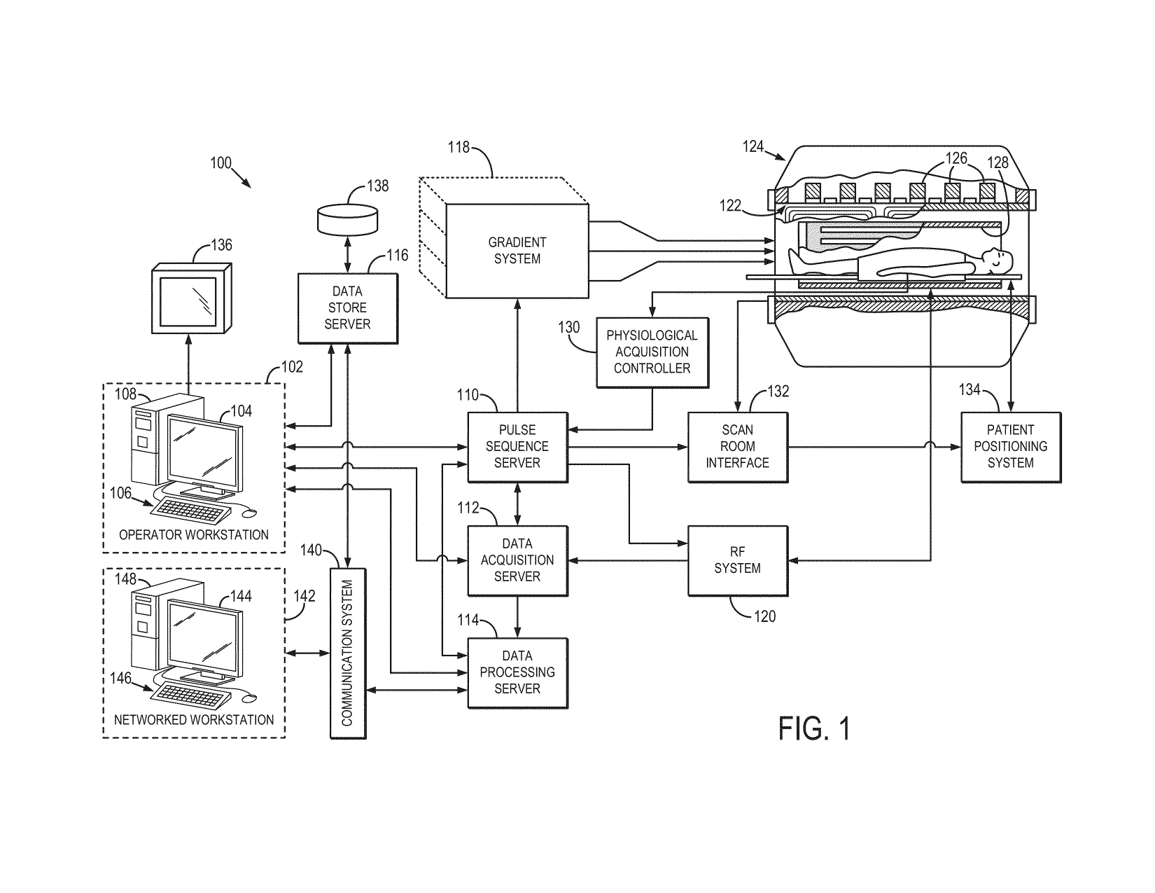

Referring particularly to FIG. 1, an example of a magnetic resonance imaging (MRI) system 100 is illustrated. The MRI system 100 includes a workstation 102 having a display 104 and a keyboard 106. The workstation 102 includes a processor 108 that is commercially available to run a commercially-available operating system. The workstation 102 provides the operator interface that enables scan prescriptions to be entered into the MRI system 100. The workstation 102 is coupled to four servers: a pulse sequence server 110; a data acquisition server 112; a data processing server 114; and a data store server 116. The workstation 102 and each server 110, 112, 114, and 116 are connected to communicate with each other.

The pulse sequence server 110 functions in response to instructions downloaded from the workstation 102 to operate a gradient system 118 and a radiofrequency (RF) system 120. Gradient waveforms necessary to perform the prescribed scan are produced and applied to the gradient system 118, which excites gradient coils in an assembly 122 to produce the magnetic field gradients Gₓ, G_{y}, and G_{z} used for position encoding MR signals. The gradient coil assembly 122 forms part of a magnet assembly 124 that includes a polarizing magnet 126 and a whole-body RF coil 128 or local coil.

RF excitation waveforms are applied to the RF coil 128, or a separate local coil, such as a head coil, by the RF system 120 to perform the prescribed magnetic resonance pulse sequence. Responsive MR signals detected by the RF coil 128, or a separate local coil, are received by the RF system 120, amplified, demodulated, filtered, and digitized under direction of commands produced by the pulse sequence server 110. The RF system 120 includes an RF transmitter for producing a wide variety of RF pulses used in MR pulse sequences. The RF transmitter is responsive to the scan prescription and direction from the pulse sequence server 110 to produce RF pulses of the desired frequency, phase, and pulse amplitude waveform. The generated RF pulses may be applied to the whole body RF coil 128 or to one or more local coils or coil arrays.

The RF system 120 also includes one or more RF receiver channels. Each RF receiver channel includes an RF preamplifier that amplifies the MR signal received by the coil 128 to which it is connected, and a detector that detects and digitizes the quadrature components of the received MR signal. The magnitude of the received MR signal may thus be determined at any sampled point by the square root of the sum of the squares of the I and Q components:

M=\sqrt{I²+Q²} (1);

and the phase of the received MR signal may also be determined:

$$ϕ = {tan}^{- 1} ( \frac{Q}{I} ) . \qquad ( 2 )$$

The pulse sequence server 110 also optionally receives patient data from a physiological acquisition controller 130. The controller 130 receives signals from a number of different sensors connected to the patient, such as electrocardiograph (ECG) signals from electrodes, or respiratory signals from a bellows or other respiratory monitoring device. Such signals may be used by the pulse sequence server 110 to synchronize, or “gate,” the performance of the scan with the subject's heart beat or respiration.

The pulse sequence server 110 also connects to a scan room interface circuit 132 that receives signals from various sensors associated with the condition of the patient and the magnet system. It is also through the scan room interface circuit 132 that a patient positioning system 134 receives commands to move the patient to desired positions during the scan.

The digitized MR signal samples produced by the RF system 120 are received by the data acquisition server 112. The data acquisition server 112 operates in response to instructions downloaded from the workstation 102 to receive the real-time MR data and provide buffer storage, such that no data is lost by data overrun. In some scans, the data acquisition server 112 does little more than pass the acquired MR data to the data processor server 114. However, in scans that require information derived from acquired MR data to control the further performance of the scan, the data acquisition server 112 is programmed to produce such information and convey it to the pulse sequence server 110. For example, during prescans, MR data is acquired and used to calibrate the pulse sequence performed by the pulse sequence server 110. Also, navigator signals may be acquired during a scan and used to adjust the operating parameters of the RF system 120 or the gradient system 118, or to control the view order in which k-space is sampled.

The data processing server 114 receives MR data from the data acquisition server 112 and processes it in accordance with instructions downloaded from the workstation 102. Such processing may include, for example: Fourier transformation of raw k-space MR data to produce two or three-dimensional images; the application of filters to a reconstructed image; the performance of a backprojection image reconstruction of acquired MR data; the generation of functional MR images; and the calculation of motion or flow images.

Images reconstructed by the data processing server 114 are conveyed back to the workstation 102 where they are stored. Real-time images are stored in a data base memory, from which they may be output to the operator display 104 or a display 136 that is located near the magnet assembly 124 for use by attending physicians. Batch mode images or selected real time images are stored in a host database on disc storage 138. When such images have been reconstructed and transferred to storage, the data processing server 114 notifies the data store server 116 on the workstation 102. The workstation 102 may be used by an operator to archive the images, produce films, or send the images via a network or communication system 140 to other facilities that may include other networked workstations 142.

The communications system 140 and networked workstation 142 may represent any of the variety of local and remote computer systems that may be included within a given clinical or research facility including the system 100 or other, remote location that can communicate with the system 100. In this regard, the networked workstation 142 may be functionally and capably similar or equivalent to the operator workstation 102, despite being located remotely and communicating over the communication system 140. As such, the networked workstation 142 may have a display 144 and a keyboard 146. Also, the networked workstation 142 may be a mobile device, including a laptop, phone, or tablet, that has corresponding user interfaces and, in some cases, reduced control and functionality compared to the operator workstation 102. The networked workstation 142 includes a processor 148 that is commercially available to run a commercially-available operating system. The networked workstation 142 may be able to provide the operator interface that enables scan prescriptions to be entered into the MRI system 100.

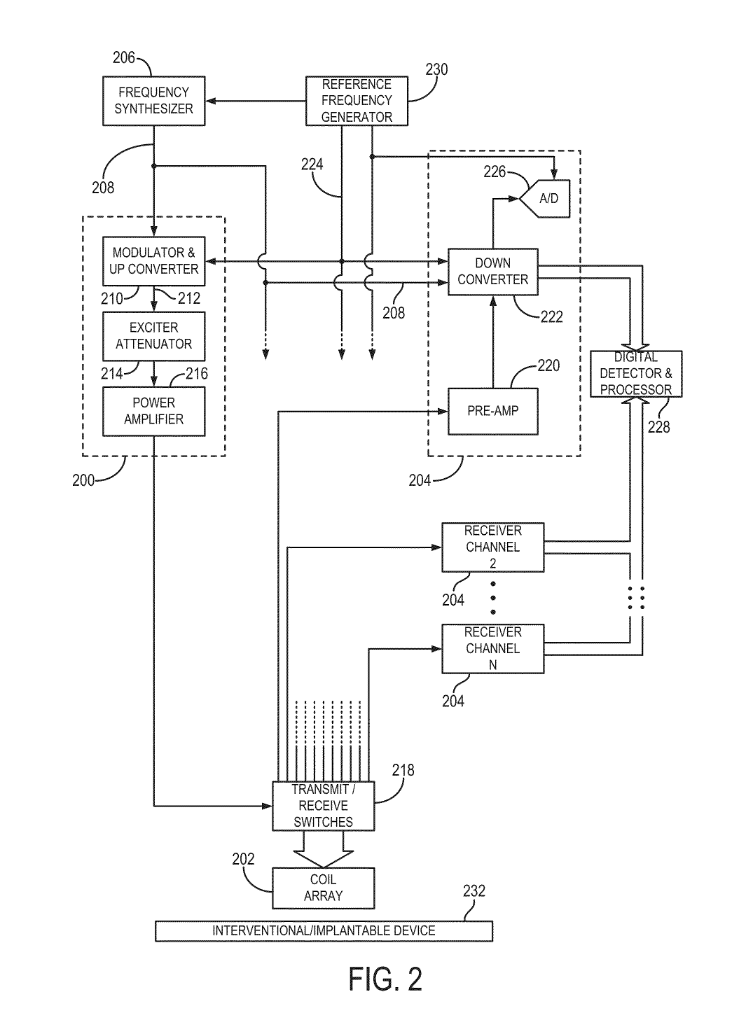

As shown in FIG. 1, the RF system 120 may be connected to the whole-body RF coil 128, or as shown in FIG. 2, a transmitter section of the RF system may connect to at least one transmit channel 200 of a coil array 202, and a receiver section of the RF system 120 may connect to at least one receiver channel 204 of the coil array 202. Often, the transmitter section is connected to the whole-body RF coil or a local transmit coil. Often, at least one receiver channel 204 is connected to the whole-body RF coil, or to one or more local receive coils which may also include the whole-body RF coil. In so-called “parallel receiver” coil arrays, one receiver channel 204 is connected to each receive coil element of the coil array 202.

Referring particularly to FIG. 2, the RF system includes a transmitter 200 that produces a prescribed RF excitation field. The base, or carrier, frequency of this RF excitation field is produced under control of a frequency synthesizer 206 that receives a set of digital signals from the pulse sequence server 110. These digital signals indicate the frequency and phase of the RF carrier signal produced at an output 208. The RF carrier is applied to a modulator and up-converter 210 where its amplitude is modulated in response to a signal, R(t), also received from the pulse sequence server 110. The signal, R(t), defines the envelope of the RF excitation pulse to be produced and is produced by sequentially reading out a series of stored digital values. These stored digital values may be changed to enable any desired RF pulse envelope to be produced.

The magnitude of the RF excitation pulse produced at output 212 is attenuated by an exciter attenuator circuit 214 that receives a digital command from the pulse sequence server 118. The attenuated RF excitation pulses are applied to a power amplifier 216, which drives the RF coil array 202 through a transmit/receive (T/R) switch 218.

Referring still to FIG. 2, the signal produced by the subject is picked up by the coil array 202 and applied to the inputs of a set of receiver channels 204. A pre-amplifier 220 in each receiver channel 204 amplifies the signal by an amount determined by a digital attenuation signal received from the pulse sequence server 110. The received signal is at or around the Larmor frequency, and this high-frequency signal is down-converted in a two step process by a down converter 222, which first mixes the detected signal with the carrier signal on line 208 and then mixes the resulting difference signal with a reference signal on line 224. The down-converted MR signal is applied to the input of an analog-to-digital (A/D) converter 226 that samples and digitizes the analog signal and applies it to a digital detector and signal processor 228 that produces 16-bit in-phase (I) values and 16-bit quadrature (Q) values corresponding to the received signal. The resulting stream of digitized I and Q values of the received signal are output to the data acquisition server 112. The reference signal, as well as the sampling signal applied to the ND converter 226, are produced by a reference frequency generator 230.

As described, when the RF system 120 is used in conjunction with or proximate to an interventional or implantable (or implanted) device 232, additional care and planning is taken to protect against or control undesired heating, or to control desired heating. Unwanted heating may include any heating of the device 232 or may include uncontrolled or undesired heating, for example, in the case of RF ablation or coagulation. That is, in the case of an imaging procedure, any heating or any heating above some minimal level may be undesirable. On the other hand, in some procedures, such as MR ablation or MR coagulation procedures, some controlled heating may be desired.

In MR RF ablation and MR RF coagulation it is desired to induce RF heating at the tip of a guidewire during an interventional procedure carried out within an MRI scanner. The heating is optimized if the guidewire is resonant at the scanner operating frequency, but it is difficult to determine if the guidewire is resonant because the resonant frequencies will change as the guidewire is advanced or otherwise manipulated. The resonant frequencies will change dynamically in response to many factors such as the length, position, and geometrical configuration of the guidewire, and the nature of the materials and tissues which it contacts.

The systems and methods provided in the present disclosure overcomes these challenges. As will be described, the above-described MR system 100 can be configured in accordance with the present disclosure to provide systems and methods to measure the resonant frequencies of an interventional or implantable (or implanted) device 232 or a wire-like structure within the human body, regardless of the specific configuration of the wire. Furthermore, the systems and methods of the present disclosure allows the MR system 100 and the user or clinician to alter the resonant frequencies of the device 232 to control resonance of the device 232 with the RF field of an MRI scanner. Accordingly, the systems and methods allow improved and active control of heating of the device 232. In MR thermal ablation and MR thermal imaging, these systems and methods can be used to monitor the resonant frequencies and either maximize or suppress heating as desired during the procedure. The device 232 can be a catheter, guidewire, wire lead, or other device, including implantable devices, such as pacemakers, orthopedic wires, and the like. The systems and methods provided herein allow the device 232 to retain the normal mechanical, handling, and functional characteristics of the device 232, and preserves low frequency electrical characteristics. Furthermore, the systems and methods described herein can be applied to MRI scanners of any field strength, and to MRI scanners that operate at multiple frequencies (for example while conducting multinuclear imaging or spectroscopy studies).

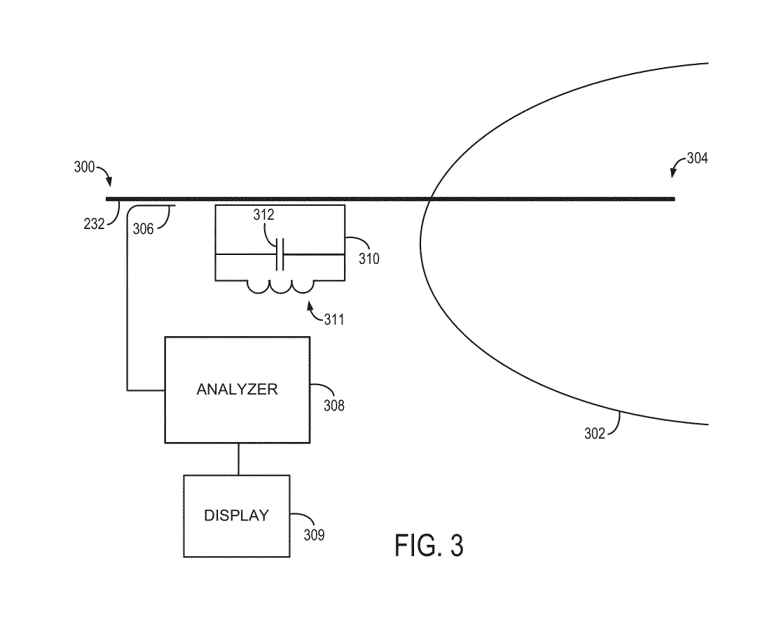

Referring to FIG. 3, a device 232, may be assumed to have the form of long wire structure and may be modeled as either a transmission line or an antenna extending from a proximal end 300, which may extend outside a subject's body 302 to a distal end 304 within the subject's body 302. The device 232 may be coupled to an analyzing probe 306 that is connected to an analyzer system 308. The probe 306 may be a directional coupler or other device that samples the RF power on the device 232 without requiring a direct electrical connection to the device 232, or the probe 306 may be an electrical network electrically connected to the device 232. The analyzer system 308 may be a network analyzer that includes or is coupled to a display 309. The analyzing probe 306, which is connected to or electromagnetically coupled to the device 232, provides feedback to the network analyzer system 308, which may be a network analyzer that displays electrical properties of a connected circuit as a function of frequency. As will be described, the display 309 may be one of the displays of the MRI system 100, such that the analyzer system 308 provides feedback to the MRI system 100 or other computer control system regarding the RF-induced current flowing in the device 232.

A resonant frequency adjustment device 310, which includes a dual directional coupler, a transformer, or other electrical circuit that does not require direct electrical connection to the device 232, may be included. The resonant frequency adjustment device 310 may include the ability to adjust the electrical configuration of the device 232. As a non-limiting example the resonant frequency adjustment device 310 may include a coil 311 and capacitor 312. The resonant frequency adjustment device 310 may include a variety of combinations of coils, capacitors, and resistors or other components to assist in influencing or adjusting the electrical properties and, more particularly, the resonant frequencies of the device 232. The resonant frequency adjustment device 310 may connect to the device 232 with a direct electrical connection or may couple to the device 232 with a dual directional coupler, a transformer, or other electrical circuit that does not require direct electrical connection.

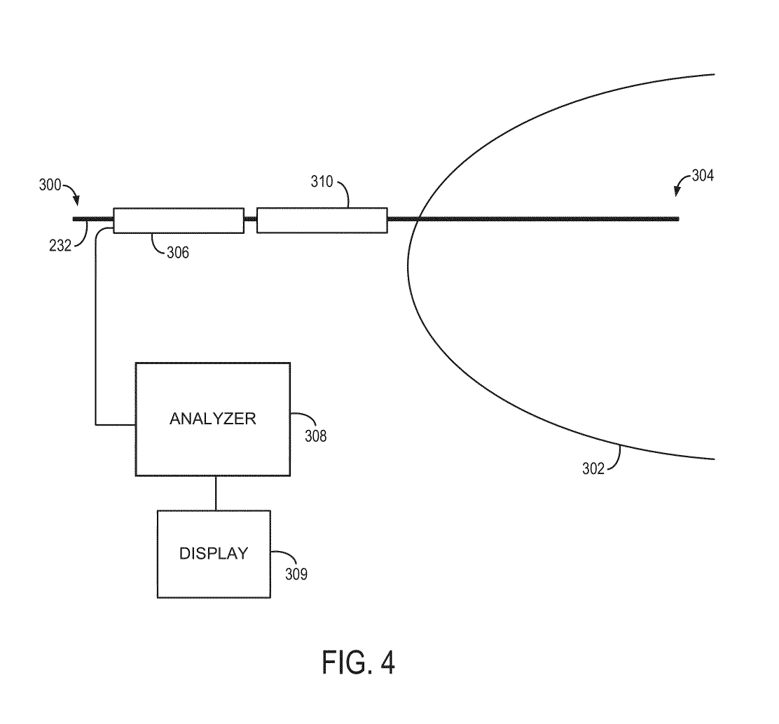

As illustrated in FIG. 4, the analyzer 308 may be coupled to the device 232 by the probe 306. Alternatively, the probe 306 may be slipped over or otherwise arranged in parallel with or proximate to the device 232 and, in a similar manner, the resonant frequency adjustment device 310 may be slipped over the device 232, such as illustrated in FIG. 4. Also, the coupler 308 and/or directional coupler 310 may be connected to the device 232 through direct electrical connection instead of indirect coupling.

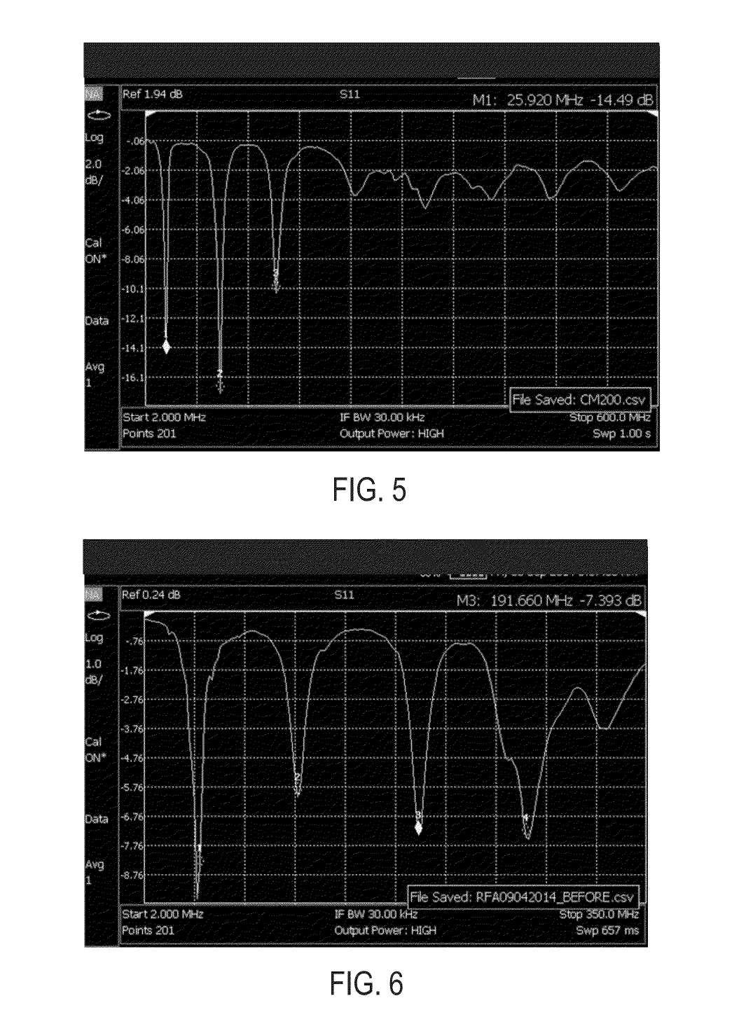

As described above with respect to FIGS. 3 and 4, the analyzer system 308 may be a network analyzer that includes a display 309 and is connected via the directional coupler to the device 232. The analyzer system 308 acquires data that can be communicated via, for example, the display 309 to control the resonance of the device 232. For example, referring now to FIG. 5, a graph can be provided that represents the RF current traveling in device as sampled by the probe 306. More specifically, as illustrated in FIG. 5, the graph can show the log of the magnitude of the RF power flowing in the device 232. That is, FIG. 5 shows a graph of the log magnitude reflected power flowing in a guidewire partially inserted into a normal saline solution as a function of frequency over the range of 2-600 MHz, which includes all clinically relevant MRI scanner operating frequencies. Note the dip in reflected power seen at the resonant frequencies of this particular guidewire configuration.

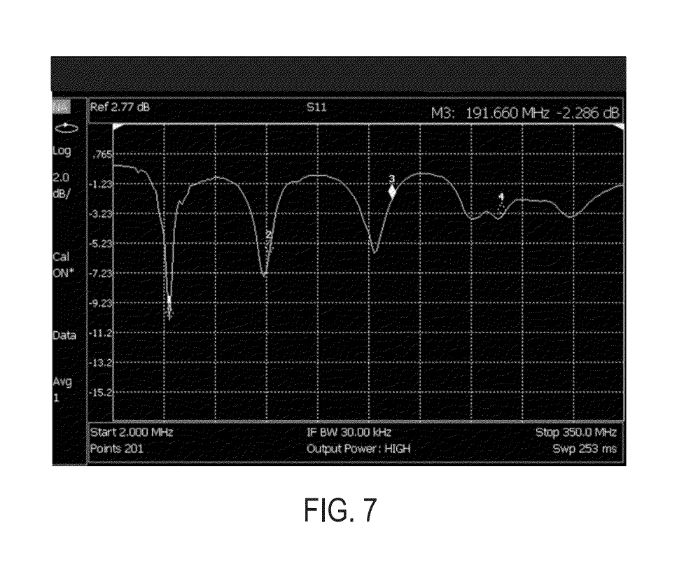

As best illustrated in FIGS. 6 and 7, frequencies at which the device is resonant exhibit an extreme value of reflected power. Since the resonant length of a device is dependent on many variables in addition to its length, and can change as the device's geometrical configuration (e.g., its shape) is changed, or as it comes in contact with various substances (e.g., a variety of tissues, the catheter, air), it is very difficult to predict its resonant frequencies. However, using the probe 306, in this example implemented as a directional coupler, connected to an analyzer permits the resonant frequencies to be precisely measured during the interventional surgical procedure, or when the medical device is being implanted. In particular, FIG. 6 shows the log magnitude reflected power of a guidewire partially inserted into a normal saline solution. FIG. 7 shows the log magnitude reflected power of the same guidewire inserted into the normal saline solution but after the frequency adjustment device 310, implemented in this example as an inductor inserted in series with the guidewire, is included to knowingly alter the resonant frequencies.

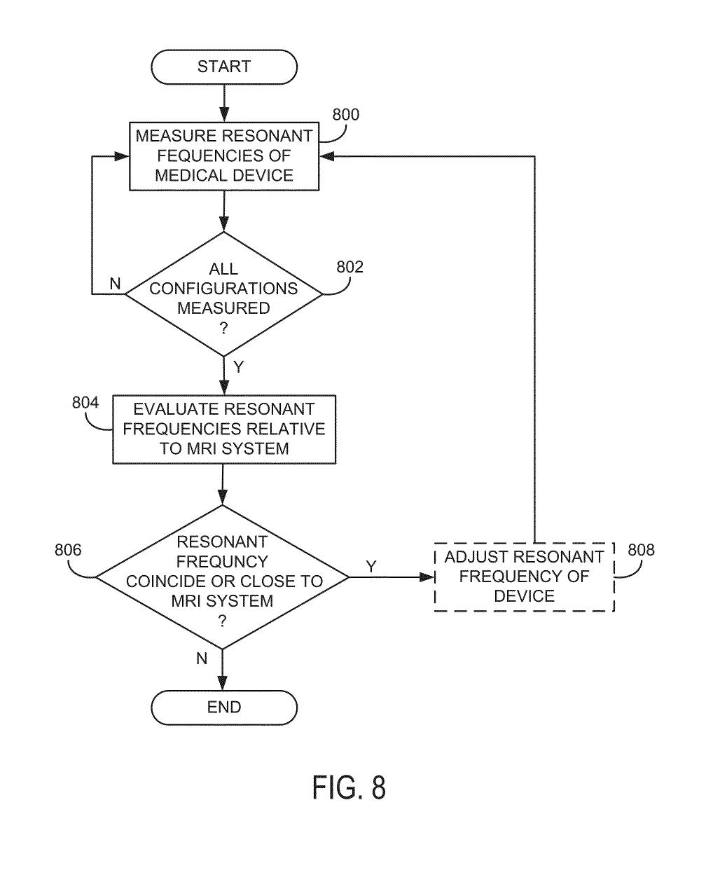

Referring to FIG. 8, an example of steps of a process for controlling a resonant frequency of an implantable or interventional medical device is illustrated. The process starts by measuring the resonant frequencies of a given medical device at process block 800. To do so, an analyzing system, such as described above can be used to determine for each and every configuration (or all desired or practical configurations) of the medical device 232. Once all configurations and resonant frequencies have been measured at decision block 802, the resonant frequencies can be evaluated to determine whether some may coincide with or be sufficiently close to that of an MRI scanner operating frequency at process block 804. At decision block 806, if a resonant frequency of the medical device is close to that of a desired an MRI scanner operating frequency, feedback is provided to the user or clinician at process block 808 that there is a risk of RF heating of the device or a portion of the device, and the device should therefore be considered unsafe for use within an MRI scanner operating at that frequency.

If the device is determined to have a resonant frequency that could be problematic with respect to the MRI scanner, optionally, the resonant frequencies of the device may be altered to control against coincidence with the operating frequency of the MRI scanner. To this end, the frequency adjustment device 310 described above with respect to FIGS. 3 and 4 may be used to adjust the resonant frequency of the medical device. For example, the if the medical device is a catheter or guidewire or wire lead, the frequency adjustment device 310 may be connected thereto to form an electrical circuit that alters the resonant frequencies of the medical device. In this non-limiting example of a catheter, guidewire, or wire lead, the coupler and electrical circuit may be used to alter the electrical length of the elongated portion of the device, thereby modifying its resonant frequencies.

Thus, referring again to FIG. 8, at optional process block 808, the resonant frequencies of the device may be altered such that resonance with an operating frequency of the MRI scanner is avoided, thereby reducing or controlling the heating effect. Thus, for example, despite potentially unpredictable changes in the resonant frequencies of a guidewire as it is inserted and manipulated during an interventional surgical procedure carried out in an MRI scanner, the surgeon can monitor and then alter as needed the wire resonant frequencies to ensure that they never overlap with an operating frequency of the MRI scanner, thereby reducing the possibility of burning the patient.

As described above, the device may be functionally connected to the resonant frequency adjustment device 310 that is designed to adjust the resonant frequency of the device to which it is functionally connected or coupled. For example, the resonant frequency adjustment device 310 may include a directional coupler or other coupling. Alternatively, the resonant frequencies of the device may be modified or altered by connecting an electrical circuit directly to the wire instead of coupling to it with a coupler. As described above, FIG. 7 shows an example of a guidewire in which the resonant frequencies are modified by placing an inductor in series with a guidewire. That is, FIG. 6 shows the spectrum of the resonant frequencies of the guidewire itself and FIG. 7 shows the spectrum following insertion of the series inductor.

As described above, in many clinical settings, RF-induced heating of an implanted or interventional medical device due to an MR system is undesirable and may be controlled or minimized. However, in the case of MR thermal ablation and MR thermal coagulation, the above-described systems and methods can be used to adjust the resonant frequencies of the implantable/interventional device, such as a needle or guidewire, to ensure that at least one resonant frequency coincides with the operating frequency of the MRI scanner to, thereby, effectuate the desired heating of the needle or guidewire tip, as desired for the thermal ablation or coagulation process. Additionally, the above-described systems and methods can be used to continuously monitor the resonant frequencies of a device to increase or maximize the heating effect when desired while ablation or coagulation is being conducted, and to attenuate the heating effect at other times, for example when the scanner is operated to view the result of the ablation or coagulation, or to track the positioning of the guidewire into the target lesion.

Further variations on the above-described systems and methods are possible. For example, the analyzer system may include a network analyzer that allows the user to determine the resonant frequencies of the device and make adjustments accordingly. Other systems besides a network analyzer can also be used to make the measurement, for example an inexpensive, compact, MRI-compatible special purpose measurement device specifically designed for determining the resonant frequencies. However, the process of measuring and then altering the resonant frequencies may be automated by an electronic or computerized device such that the process occurs automatically without operator intervention. In this case, the process described with respect to FIG. 7 may be controlled by a computer system that may or may not be integrated with an imaging system, such that a processor or controller carries out the steps set forth in FIG. 7.

In still other implementations, the circuit that alters the resonant frequencies may be comprised of a capacitor, an inductor, a resistor, or a combination of these circuit elements. In still other implementations, the circuit that alters the resonant frequencies may be coupled to the catheter, guidewire, or wire lead with a coupler or a transformer, or may be directly connected, or may employ a combination of these methods. In yet other configurations, the catheter, guidewire, or wire lead may be directly connected to the network analyzer or other measurement device.

The present invention has been described in terms of one or more preferred embodiments, and it should be appreciated that many equivalents, alternatives, variations, and modifications, aside from those expressly stated, are possible and within the scope of the invention.

1. A system configured to be utilized with a magnetic resonance imaging (MRI) system to facilitate use of a medical device during operation of the MRI system, the system comprising:
a probe configured to be electrically coupled to a medical device configured to be inserted or implanted into a subject;
an analyzer system configured to receive data from the probe and determine resonant frequencies of the medical device using the data; and
a display configured to generate a report of the resonant frequencies of the medical device at least over predicted operating frequencies of the MRI system during an imaging procedure using the MRI system and in the presence of the medical device and indicating any resonant frequencies of the medical device coincident with the predicted operating frequencies of the MRI system during the medical procedure.

2. The system of claim 1 further comprising a resonant frequency adjustment device configured to adjust the resonant frequencies of the medical device.

3. The system of claim 2 wherein the resonant frequency adjustment device includes at least one of a coil, a capacitor, a resistor, or a combination of electrical components configured to change electrical properties of the medical device.

4. The system of claim 3 wherein the electrical properties include at least one of reflected power or impedance.

5. The system of claim 3 wherein the resonant frequency adjustment device includes a directional coupler.

6. The system of claim 2 further comprising a processor configured to receive the report and control operation of the resonant frequency adjustment device to adjust the resonant frequencies of the medical device to be one of matched or unmatched to the predicted operating frequencies of the MRI system.

7. The system of claim 1 wherein the medical device includes at least one of a catheter, a guidewire, a lead wire, an ablation probe, or a coagulation probe.

8. The system of claim 1 wherein the analyzer system includes a network analyzer.

9. A system configured to be utilized with a magnetic resonance imaging (MRI) system to facilitate use of a medical device during operation of the MRI system, the system comprising:
a probe configured to be electrically coupled to a medical device configured to be inserted or implanted into a subject to perform a medical procedure;
an analyzer system configured to receive data from the probe and determine resonant frequencies of the medical device using the data;
a processor configured to:
determine the resonant frequencies of the medical device;
compare the resonant frequencies of the medical device to predicted operating frequencies of the MRI system during the medical procedure; and
upon determining a commonality of the resonant frequencies of the medical device during the medical procedure and the predicted operating frequencies of the MRI system, adjust the resonant frequencies of the medical device to reduce the commonality of the resonant frequencies of the medical device during the medical procedure and the predicted operating frequencies of the MRI system.

10. The system of claim 9 further comprising a resonant frequency adjustment device configured to be controlled by the processor to adjust the resonant frequencies of the medical device.

11. The system of claim 10 wherein the resonant frequency adjustment device includes at least one of a coil, a capacitor, a resistor, or a combination of electrical components configured to change electrical properties of the medical device.

12. The system of claim 11 wherein the electrical properties include at least one of reflected power or impedance.

13. The system of claim 10 wherein the resonant frequency adjustment device includes a directional coupler.

14. The system of claim 9 wherein the analyzer system includes a network analyzer.

15. A method for performing an image guided interventional medical procedure, the method including steps comprising:
i) electrically analyzing a medical device to determine resonant frequencies of the medical device;
ii) comparing the resonant frequencies of the medical device to predicted operating frequencies of a magnetic resonance imaging (MRI) system during a planned medical procedure performed using the medical device and guided by images acquired by the MRI system; and
iii) determining a commonality of the resonant frequencies of the medical device during the medical procedure and the predicted operating frequencies of the MRI system;
iv) adjusting the resonant frequencies of the medical device to reduce the commonality of the resonant frequencies of the medical device during the medical procedure and the predicted operating frequencies of the MRI system; and
v) arranging a subject within the MRI system to acquire images of the subject during the medical procedure performed using the medical device.

16. The method of claim 15 further comprising repeating steps i) through iv) during the medical procedure.

17. The method of claim 15 wherein the medical device includes at least one of a catheter, a guidewire, a lead wire, an ablation probe, or a coagulation probe.

18. A magnetic resonance imaging (MRI) system, comprising:
a magnet system configured to generate a polarizing magnetic field about at least a portion of a subject arranged in the MRI system;
a plurality of gradient coils configured to establish at least one magnetic gradient field to the polarizing magnetic field;
a radio frequency (RF) system configured to apply an RF field to the subject during a transmit phase and receive imaging signals from the subject during a receive phase;
a processor configured to:
i) compare measured resonant frequencies of a medical device to predicted operating frequencies of the MRI system during a planned medical procedure performed using the medical device and guided by images acquired by the MRI system;
ii) determine a commonality of the resonant frequencies of the medical device during the medical procedure and the predicted operating frequencies of the MRI system;
iii) adjust the resonant frequencies of the medical device to reduce the commonality of the resonant frequencies of the medical device during the medical procedure and the predicted operating frequencies of the MRI system; and
iv) repeat i) through iii) during the medical procedure.

19. The system of claim 18 further comprising a resonant frequency adjustment device coupled to the medical device and configured to be controlled by the processor to adjust the resonant frequencies of the medical device

20. The system of claim 18 further comprising an analyzer system configured to receive data from the probe regarding electrical properties of the medical device and wherein the processor is configured to receive the data from the analyzer system and determine resonant frequencies of the medical device using the data.
